(19) United States
(12) Patent Application Publication
(10) Pub. No.: US 2015/0125484 A1
(43) Pub. Date: May 7, 2015

(54) Composition for promoting hair growth

(71) Applicants: Li Ren Wang, Taipei (TW); Hua Guan Bio-tech Corporation, Taipei (TW)

(72) Inventor: Li Ren Wang, Taipei (TW)

(73) Assignees: Li Ren Wang, Taipei (TW); Hua Guan Bio-tech Corporation, Taipei (TW)

(21) Appl. No.: 14/070,602

(22) Filed: Nov. 4, 2013

(51) Int. Cl. A61K 8/97; A61Q 7/00

(52) U.S. Cl. CPC A61K 8/975; A61Q 7/00; A61K 8/97
USPC 424/195.15

(57) ABSTRACT

A composition for promoting hair growth is provided with fermented and distilled extracts of fermented and distilled extracts of ganoderma, fungus, grapes, sorghum, wheat, onions, ginger, burdock, aloe vera, carrot, rose, litsea, tea, whip grass, incense, sage, blue citrus, bergamot, rosmarinus officinalis, sesame oil tree, nutmeg, rosewood, orange, marigold, geranium, and honey flower. The composition can be made into shampoo.

### BACKGROUND OF THE INVENTION

1. Field of the Invention

The invention relates to treatments of androgenetic alopecia and more particularly to a composition for promoting hair growth.

2. Description of Related Art

Hair loss has significant negative social and psychological effects. Demand for treating hair loss is high worldwide. There are various treatment products as well as surgical procedures for hair growth such as hair transplants. While a hair transplant process can be effective, it is very expensive.

Regarding hair loss treatment products, most of them have no positive effect at all. A few of them have shown some results. However, a continued application or treatment of the products is required. Otherwise, the improvement may be lost.

It is thus desirable to have a treatment which is effective in treating hair loss.

### SUMMARY OF THE INVENTION

It is therefore one object of the invention to provide a composition for promoting hair growth composition comprising fermented and distilled extracts of fermented and distilled extracts of ganoderma, fungus, grapes, sorghum, wheat, onions, ginger, burdock, aloe vera, carrot, rose, litsea, tea, whip grass, incense, sage, blue citrus, bergamot, rosmarinus officinalis, sesame oil tree, nutmeg, rosewood, orange, marigold, geranium, and honey flower.

The above and other objects, features and advantages of the invention will become apparent from the following detailed description taken with the accompanying drawings.

### BRIEF DESCRIPTION OF THE DRAWINGS

FIG. 1 is a perspective view of a container containing a fluid composition for promoting hair growth according to the invention;

FIG. 2 is flow chart illustrating steps of preparing the composition; and

FIG. 3 is a first photograph showing before applying the composition to the head and improvement after applying the composition to the head;

FIG. 4 is a second photograph showing before applying the composition to the head and improvement after applying the composition to the head; and

FIG. 5 is a third photograph showing before applying the composition to the head and improvement after applying the composition to the head.

### DETAILED DESCRIPTION OF THE INVENTION

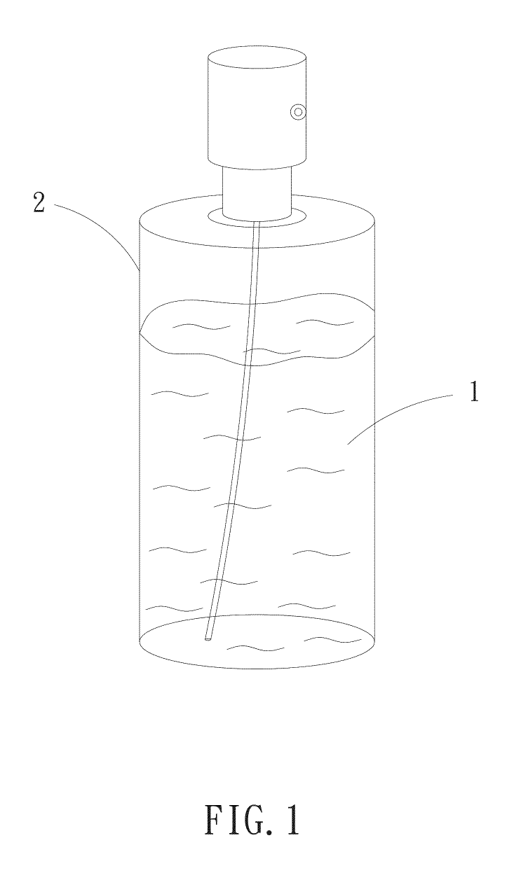

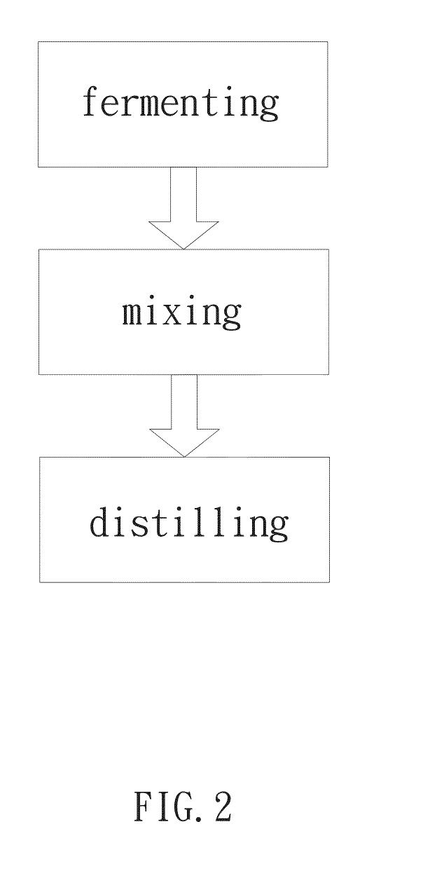

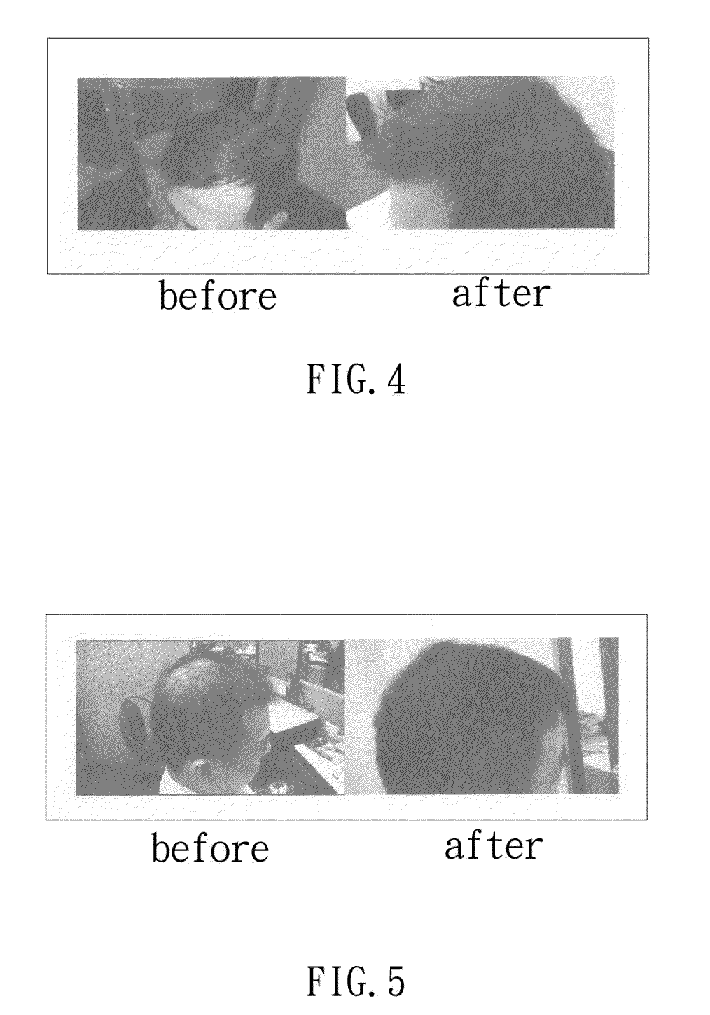

Referring to FIGS. 1 to 5, a composition 1 for promoting hair growth in accordance with the invention is prepared by fermenting extracts of ganoderma, fungus, grapes, sorghum, wheat, onions, ginger, burdock, aloe vera, carrot, rose, litsea, tea, whip grass, incense, sage, blue citrus, bergamot, rosmarinus officinalis, sesame oil tree, nutmeg, rosewood, orange, marigold, geranium, and honey flower.

The fermentation is either aerobic fermentation or anaerobic fermentation wherein pH and temperature are maintained at predetermined, optimum values. The preparation further includes steps of adding above fermented extracts into a container 2 to mix; and distilling the mixture to produce the composition 1.

Roses are obtained through the distillation process of rose extracts. Ginger is obtained from the shade of ginger. Burdock is obtained from the shade of the burdock. Radish is obtained from the shade of the radish. Aloe vera can also be replaced by a mixture of aloe vera and other materials. Ginger may also be obtained by slicing ginger strips. Burdock may also be obtained by slicing burdock. Radish may also be obtained by slicing radish. Grape extracts are fermented 7 days and have ethanol concentration of about 5-10%. Sorghum extracts are fermented 7 days and have ethanol concentration about 5-30%. Wheat extracts are fermented and have ethanol concentration about 5 to 30%.

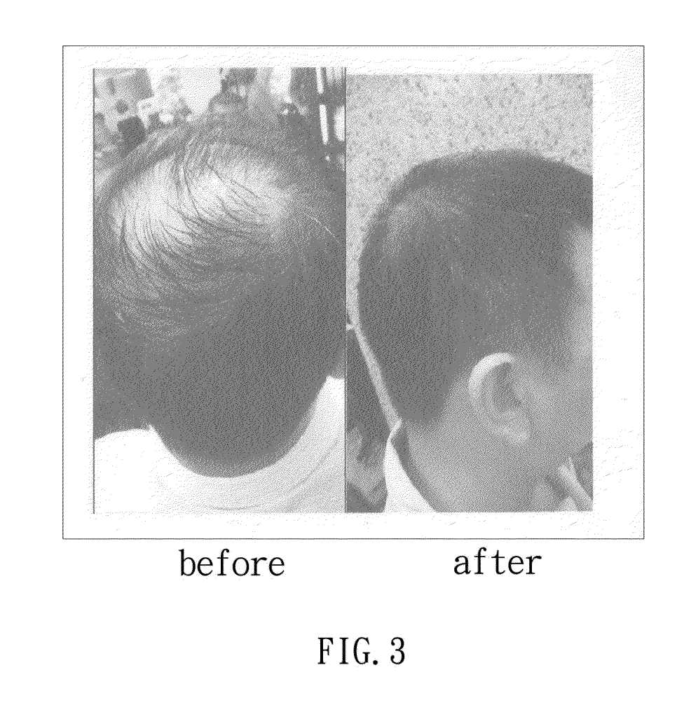

The composition 1 can be made into tonic, shampoo, conditioner, hair cream, hair gel, eyebrow maintenance fluid, eyelashes maintenance solution, ointment, lotion or mousse related product. The composition 1 can be absorbed by hair follicles in the scalp area after spraying or applying onto the head. The absorbed composition 1 can activate the internal mechanism and reach the epidermis and hair follicle cells for promoting hair growth as best demonstrated by referring to FIGS. 3 to 5.

While the invention has been described in terms of preferred embodiments, those skilled in the art will recognize that the invention can be practiced with modifications within the spirit and scope of the appended claims.

What is claimed is:

1. A composition for promoting hair growth comprising:
fermented and distilled extracts of ganoderma, fungus, grapes, sorghum, wheat, onions, ginger, burdock, aloe vera, carrot, rose, litsea, tea, whip grass, incense, sage, blue citrus, bergamot, rosmarinus officinalis, sesame oil tree, nutmeg, rosewood, orange, marigold, geranium, and honey flower.

2. The composition of claim 1, wherein the fermentation is either aerobic fermentation or anaerobic fermentation.

3. The composition of claim 1, wherein the grape extracts are fermented 7 days and the fermented grape extracts have ethanol concentration of about 5-10%, the sorghum extracts are fermented 7 days and the fermented sorghum extracts have ethanol concentration about 5-30%, and the fermented wheat extracts have ethanol concentration about 5 to 30%.
